(19) United States
(12) Patent Application Publication
(10) Pub. No.: US 2024/0392385 A1
(43) Pub. Date: Nov. 28, 2024

(54) METHOD FOR PREDICTING RESPONSE TO CANCER IMMUNOTHERAPY BY USING DNA METHYLATION ABERRATION IN LINE-1

(71) Applicants: PentaMedix Co., Ltd., Seongnam-si, Gyeonggi-do (KR); KOREA ADVANCED INSTITUTE OF SCIENCE AND TECHNOLOGY, Daejeon (KR)

(72) Inventors: Jung Kyoon CHOI, Sejong (KR); Kyeong Hui KIM, Daejeon (KR); In Kyung SHIN, Seongnam-si (KR); Seung Jae NOH, Hwaseong-si (KR); Dae Yeon CHO, Seongnam-si (KR)

(73) Assignees: PentaMedix Co., Ltd., Seongnam-si, Gyeonggi-so (KR); KOREA ADVANCED INSTITUTE OF SCIENCE AND TECHNOLOGY, Daejeon (KR)

(21) Appl. No.: 18/696,031

(22) PCT Filed: Sep. 29, 2022
(86) PCT No.: PCT/KR2022/014662

§ 371 Date: Mar. 27, 2024

(30) Foreign Application Priority Data
Sep. 29, 2021 (KR) 10-2021-0128751
Sep. 29, 2022 (KR) 10-2022-0124049

(51) Int. Cl. C12Q 1/6886

(52) U.S. Cl. CPC C12Q 1/6886; C12Q 2600/106; C12Q 2600/154

(57) ABSTRACT

The present disclosure relates to a method for providing information for predicting a response to cancer therapy, comprising the step of obtaining information on a DNA methylation level of a long interspersed nuclear element-1 (LINE-1) subfamily from an isolated sample of a cancer patient, wherein the LINE-1 subfamily is at least one selected from the group consisting of LIPA12_5end, LIHS_5end, L1PA10_3end, LIP2_5end, L1PA11_3end, L1P2_5end, L1PA13_3end, L1PA11_3end, and LIP1_orf2.

### TECHNICAL FIELD

The present disclosure relates to a method for providing information for predicting a response to immunotherapy of a cancer patient using information on a DNA methylation level of a long interspersed nuclear element-1 (LINE-1) subfamily from an isolated sample of a cancer patient.

### BACKGROUND ART

Cancer is one of the highest causes of death in the world, and the cure rate is often determined by early diagnosis, and thus early diagnosis of cancer through health checkups and the like is considered important. Generally, a cancer test commonly used during health checkups is a protein tumor marker test in the blood, and the occurrence of cancer may be confirmed through endoscopy, biopsy, etc.

Three types of abnormal mutations in a genome causing cancer are known, but may be caused by epigenetic modifications, which are chromatin modifications, in addition to changes in the order or base sequence of chromosomes, where part of the chromosome is completely changed or the base sequence is changed in 1 and 2 portions (Sticker T, Catenacci D V, Seiwert T Y. Molecular profiling of cancer—the future of personalized cancer medicine: a primer on cancer biology and the tools necessary to bring molecular testing to the clinic. Semin Oncol 2011 38 (2): 173-85). In general, mutations in which part or several locations of a chromosome are changed may be caused by genetic causes and may also occur locally in somatic cells due to mutagenic factors such as radiation. The exact cause of DNA methylation changes in cancer cells, which is the most representative of epigenetic modifications, is not known, but it is considered to be caused by diet or environmental factors. In particular, many of these epigenetic modifications are found in the genome of cancerous tissues, and an interest in clinical applications such as the development of diagnostic methods using the epigenetic modifications is increasing.

DNA methylation is a phenomenon in which a methyl group (—CH3) is added to the 5th carbon of a cytosine pyrimidine ring by covalent bonds. The DNA methylation plays an important role in various life phenomena such as genomic imprinting, X chromosome inactivation, etc. in the development of normal individuals.

In cancer tissues, two types of DNA methylation that are different from those in normal cells occur, which are global hypomethylation throughout the genome and hypermethylation of a CpG island located in a gene expression regulatory region. The hypomethylation occurs mainly in an intergenic region between genes, and is assumed to destabilize chromosomes and cause recombination, transfer, deletion, rearrangement, and the like of chromosomes during cell division. In particular, it is known that transposons such as LINE-1 are normally methylated and the expression thereof is inhibited, but due to hypomethylation in cancer, the transposons are expressed and spread throughout the genome to become a cause of chromosomal instability. Here, DNA methylation changes in cancer tissues are considered to be epigenetic, but since these epigenetic modifications are maintained even after cell division, hypomethylation and hypermethylation have a lasting effect on the expression of genes located around the transposons and CpG islands. In fact, it is known that the expression of tumor suppressor genes, cell cycle control genes, DNA repair-related genes, cell adhesion-related genes, etc. is inhibited by DNA methylation in cancer tissues to exhibit the same effect as if these genes are broken (McCabe M T, Brandes J C. Vertino P M. Cancer DNA methylation: molecular mechanisms and clinical implications. Clin Cancer Res. 2009 15 (12): 3927-37). When the expression of these genes is inhibited, the cells proliferate abnormally and are unable to maintain genetic stability, causing additional mutations and playing an important role in the progression of cancer.

Meanwhile, unlike existing anticancer drugs that target cancer cells or cancer-related genes, anti-cancer immunotherapy such as CTLA-4 and PD-1/PD-L1 immune checkpoint inhibitors activates the in-vivo immune system and helps immune cells attack cancer cells. Only a small number of patients have near-cure effects compared to the high price of the anti-cancer immunotherapy, so that it is important to select a patient group suitable for immunotherapy. However, knowledge of factors predicting a response to treatment is very limited, and there is no research on a correlation between a response to immunotherapy and DNA methylation aberration.

### DISCLOSURE

### Technical Problem

The present disclosure has been made in an effort to provide information for predicting a response to cancer therapy of a patient using information on a DNA methylation level of a long interspersed nuclear element-1 (LINE-1) subfamily from an isolated sample of the patient.

However, the aspects of the embodiments are not limited to the above-mentioned aspects, and other aspects not mentioned can be clearly understood by those skilled in the art from the following description.

### Technical Solution

An embodiment of the present disclosure provides a method for providing information for predicting a response to cancer therapy including obtaining information on a DNA methylation level of a long interspersed nuclear element-1 (LINE-1) subfamily from an isolated sample of a cancer patient.

Here, the LINE-1 subfamily may be at least one selected from the group consisting of LIPA12_5end, LIHS_5end, LIPA10_3end, LIP2_5end, LIPA11_3end, LIP2_5end, LIPA13_3end, LIPA11_3end and LIP1_orf2.

According to an embodiment, the obtaining of the information on the DNA methylation level of the LINE-1 subfamily from the isolated sample of the cancer patient includes isolating DNA from the sample: treating a compound that modifies a cytosine base of the isolated DNA: amplifying the LINE-1 subfamily using a primer binding to the modified DNA sequence: detecting methylated and unmethylated DNAs of the LINE-1 subfamily; and calculating an estimate of the methylation level of the LINE-1 subfamily using a ratio between methylated and unmethylated DNAs.

An estimate of the methylation level of the LINE-1 subfamily may be represented as a beta value, and the beta value may represent the degree of methylation in the range of 0 to 1. For example, 0 means that the corresponding CpG locus is not completely methylated, and 1 means that the corresponding CpG locus is completely methylated.

According to an embodiment, when there are several LINE-1 subfamilies, the method includes calculating a score by multiplying the estimate of each methylation level by a weight coefficient value and then adding the values.

According to an embodiment, the treating of the compound that modifies the cytosine base of DNA may include treating bisulfite but is not limited thereto.

According to an embodiment, the detecting of the methylated and unmethylated DNAs includes performing next generation sequencing.

According to an embodiment, the cancer therapy is cancer immunotherapy.

According to an embodiment, the sample is cell free DNA.

According to an embodiment, the cancer is one selected from the group consisting of melanoma, bladder cancer, esophageal cancer, glioma, adrenal cancer, sarcoma, thyroid cancer, colorectal cancer, prostate cancer, head and neck cancer, urothelial cancer, stomach cancer, pancreatic cancer, liver cancer, testicular cancer, ovarian cancer, endometrial cancer, cervical cancer, brain cancer, breast cancer, kidney cancer, and lung cancer.

In the present disclosure, the term ‘the method for providing the information for predicting the response to cancer therapy’ refers to providing objective basic information required for the diagnosis of cancer as a preliminary step for diagnosis, and the clinical judgment or opinion of a doctor is excluded.

In the present disclosure, the ‘methylation’ refers to a phenomenon in which a methyl group (—CH3) is covalently attached to the 5th carbon of the cytosine pyrimidine ring constituting DNA.

In the present disclosure, the ‘primer’ refers to an oligonucleotide including a region complementary to a sequence of at least six consecutive nucleotides of a target nucleic acid molecule (e.g., a target gene).

In the present disclosure, the ‘next generation sequencing (NGS)’ is a high-speed analysis method of a base sequence of the genome and is characterized by processing a large number of DNA fragments in parallel unlike existing base sequencing methods and may decode a large capacity of genomic data within a short period of time.

In the present disclosure, the ‘cell-free DNA (cfDNA)’ is defined as a nucleic acid that travels through body fluids such as bloodstream or urine and may include circulating tumor DNA (ctDNA) derived from a circulating tumor cell (CTC).

The methylation level may be measured by a method such as polymerase chain reaction (PCR) or methylation-specific polymerase chain reaction (MSP) after restriction enzyme cleavage, real time methylation-specific polymerase chain reaction (PCR), PCR using methylated DNA-specific binding protein, pyrosequencing, Methylation-Sensitive High-Resolution Melting Analysis (MS-HRM), methylation specific-high-resolution melting curve analysis and measurement of methylation using methylation-sensitive restriction enzyme, automated base analysis such as DNA chip and bisulfite sequencing, etc., but is not limited thereto.

The sample may be cell free DNA containing patient DNA. In addition, the patient DNA may be used with gDNA and the like extracted from the blood, tissue, FFPE, etc. of the patient, but is not limited thereto.

The cancer therapy is cancer immunotherapy and may include an immune checkpoint inhibitor, an immune cell therapy, a therapeutic antibody, etc. The immune checkpoint inhibitor is a drug that activates T cells to attack cancer cells by blocking the activation of an immune checkpoint protein involved in T cell inhibition, and may include CTLA-4, PD-1, and PD-L1 antibodies.

### Advantageous Effects

According to embodiments of the present disclosure, by the method for providing the information for predicting the response to cancer therapy, it is possible to provide information related to a response to cancer therapy of a patient with high accuracy.

According to embodiments of the present disclosure, by the method for providing the information for predicting the response to cancer therapy, it is possible to reduce unnecessary therapy and reduce side effects and treatment costs by selecting a patient group predicted to have good treatment effect and prognosis before proceeding with cancer therapy.

It should be understood that the effects of the present disclosure are not limited to the effects described above but include all effects that can be deduced from the detailed description of the present disclosure or configurations of the disclosure described in claims.

### DESCRIPTION OF DRAWINGS

FIG. 1 illustrates a result of survival analysis (probability) of a methylation value of a LINE-1 subfamily according to an embodiment of the present disclosure.

FIG. 2 illustrates ratios of patients having high and low methylation values of the LINE-1 subfamily according to an embodiment of the present disclosure, in a responder group and a non-responder group to cancer immunotherapy.

FIG. 3 illustrates a result of survival analysis of methylation values of 10 randomly selected targets in LINE-1.

FIG. 4 illustrates ratios of patients having high and low methylation values of 10 randomly selected targets in LINE-1, in a responder group and a non-responder group to cancer immunotherapy.

FIG. 5 illustrates p-values for survival analysis and prediction of a response to cancer immunotherapy for 10 randomly selected targets in LINE-1 and each LINE-1 subfamily according to an embodiment of the present disclosure.

FIG. 6 illustrates a distribution of methylation values in a responder group and a non-responder group. For the LINE-1 subfamily according to an embodiment of the present disclosure, the left side illustrates methylation values obtained by an Infinium Methylation 450k microarray method, and the right side illustrates a result of confirming iMethyl scores obtained by an NGS method.

FIG. 7 is a result of analyzing a difference in survival probability between a responder group and a non-responder group to cancer immunotherapy in lung cancer patients by methylation values (left) obtained by an Infinium Methylation 450k microarray method and iMethyl scores (right) obtained by an NGS method for a LINE-1 subfamily according to an embodiment of the present disclosure, respectively.

FIG. 8 illustrates results of comparing a difference in survival probability between a responder group and a non-responder group to cancer immunotherapy predicted by various factors predicting a response to immunotherapy in lung cancer patients.

FIG. 9 illustrates a result (left) of analyzing a difference in survival probability between a responder group and a non-responder group to cancer immunotherapy predicted through iMethyl scores for a LINE-1 subfamily according to an embodiment of the present disclosure from DNA extracted from a lung cancer patient sample and a ROC curve (right) for evaluating prediction performance of a response to cancer immunotherapy.

FIG. 10 illustrates a distribution of methylation values of a LINE-1 subfamily according to an embodiment of the present disclosure with respect to blood (PBMC), cell free DNA (cfDNA), and an FFPE specimen (Tissue) of tumor tissue of a breast cancer patient.

FIGS. 11A and 11B illustrate results of a correlation between blood and an FFPE specimen of tumor tissue (FIG. 11A) and results of a correlation between cfDNA and an FFPE specimen of tumor tissue (FIG. 11B) with respect to 10 LINE-1 subfamilies according to an embodiment of the present disclosure.

FIG. 12 is a principal component analysis (PCA) result for a LINE-1 subfamily according to an embodiment of the present disclosure.

FIGS. 13A to 13C illustrate results of hierarchical clustering for a LINE-1 subfamily according to an embodiment of the present disclosure.

FIG. 14 illustrates a result of analyzing a difference in survival probability between a responder group and a non-responder group to cancer immunotherapy predicted through iMethyl scores for a LINE-1 subfamily according to an embodiment of the present disclosure from cfDNA extracted from patient samples of lung cancer or breast cancer.

FIG. 15 illustrates results of comparing p-value distributions of survival analysis for a LINE-1 subfamily according to an embodiment of the present disclosure between cfDNA and tumor tissue extracted from patient samples of lung cancer or breast cancer.

FIG. 16 illustrates results of comparing p-value distributions of survival analysis by weighting iMethyl scores for a LINE-1 subfamily according to an embodiment of the present disclosure from cfDNA extracted from patient samples of lung cancer or breast cancer.

### DESCRIPTION OF MAIN REFERENCE NUMERALS OF DRAWINGS

Hereinafter, embodiments will be described in detail with reference to the accompanying drawings. However, since various modifications may be made to embodiments, the scope of the present disclosure is not limited or restricted by these embodiments. It should be understood that all modifications, equivalents and substitutes for embodiments are included in the scope of the present disclosure.

The terms used in the embodiments are used for the purpose of description only and should not be construed to be limited. The singular expression includes the plural expression unless the context clearly dictates otherwise. In the present specification, it should be understood that term “comprising” or “having” indicates that a feature, a number, a step, an operation, a component, a part or the combination thereof described in the specification is present but does not exclude a possibility of presence or addition of one or more other features, numbers, steps, operations, components, parts or combinations thereof, in advance.

Unless otherwise contrarily defined, all terms used herein including technological or scientific terms have the same meanings as those generally understood by a person with ordinary skill in the art to which embodiments pertain. Terms which are defined in a generally used dictionary should be interpreted to have the same meaning as the meaning in the context of the related art and are not interpreted as an ideal meaning or excessively formal meanings unless clearly defined in the present application.

In describing the embodiments, a detailed description of related known technologies will be omitted if it is determined that they unnecessarily make the gist of the embodiments unclear.

Hereinafter, a preferred Example is presented in order to assist understanding of the present disclosure. However, the following Examples are just provided to more easily understand the present disclosure and contents of the present disclosure are not limited by Examples.

### Example 1. Selection of LINE-1 subfamily

To confirm the methylation level within a LINE-1 factor, 10 targets in the LINE-1 factor were selected as shown in Table 1 below. In Table 1 below, Array ID was a probe ID in an Infinium Methylation 450k microarray, and RepeatMasker was information mapped from a young LINE-1 subfamily provided by the RepeatMasker database (www.repeatmasker.org). In the following Examples, the LINE-1 subfamilies were named T1 to T10 in the order shown in Table 1.

TABLE 1

| ID | Array ID | Repeat Masker |
|---|---|---|
| T1 | cg00789198 | L1PA12_5end |
| T2 | cg12678329 | L1HS_5end |
| T3 | cg17952114 | L1PA10_3end |
| T4 | ch.11.24196551F | L1P2_5end |
| T5 | cg08925882 | L1PA11_3end |
| T6 | cg05128056 | L1PA11_3end |
| T7 | ch.2.195648145F | L1P2_5end |
| T8 | cg11996397 | L1PA13_3end |
| T9 | cg01291593 | L1PA11_3end |
| T10 | ch.11.41625527F | L1P1_orf2 |

To confirm whether the 10 selected subfamilies actually had a significant effect on predicting the prognosis of cancer immunotherapy, survival analysis was first performed on 141 lung cancer patients. First, one representative value was obtained from the methylation values for the 10 subfamilies. Specifically, in order to minimize scale differences for each subfamily, Min-Max Normalization was performed on the methylation values of all patients. Then, the average of 10 normalized methylation values for each patient was calculated and used as a representative value. Thereafter, each patient was divided into a high methylation (representative value above average) or low methylation (representative value below average) group based on the average of the representative values of the entire patient groups. The survival analysis was performed using a Kaplan-Meier model.

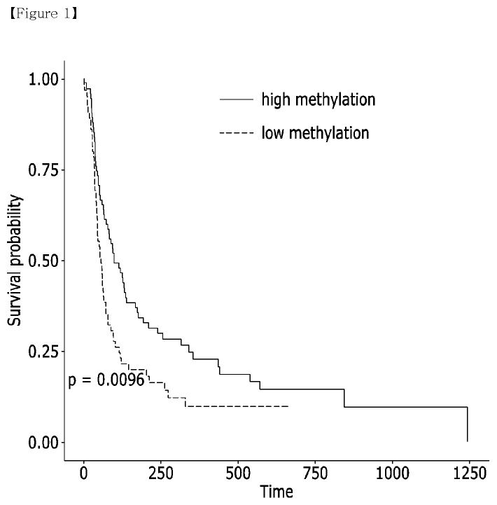

As a result, as shown in FIG. 1, it was confirmed that the high methylation group had a higher survival probability than the low methylation group, and the p-value was 0.0096, proving statistical significance. It was confirmed that the prognosis of cancer immunotherapy was well predicted only with a selected target probe set.

Next, in order to evaluate the prediction performance of a treatment response to cancer immunotherapy, the patient groups were again divided into a responder (DCB, durable clinical benefit) and a non-responder (NCB, no clinical benefit). In addition, the ratios of patients with high methylation (methylation high) and patients with low methylation (methylation low) were calculated and compared in each of the responder group and the non-responder group.

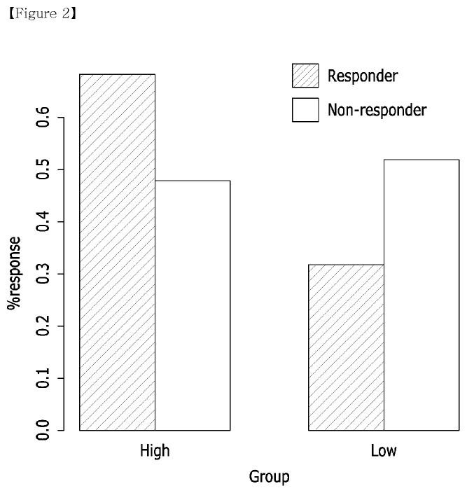

As a result, as shown in FIG. 2, in the responder group, the ratio of the patients with high methylation was higher (0.683 vs 0.480), whereas in the non-responder group, the ratio of the patients with high methylation was lower (0.317 vs 0.520), and statistical significance was also confirmed (p-value=0.026).

Through the results, it can be confirmed that the LINE-1 subfamily according to an embodiment of the present disclosure may actually predict a treatment response to cancer immunotherapy.

### Example 2. Comparison of Prediction Performance of Treatment Response of LINE-1 Subfamily and Random Targets

In order to verify whether the LINE-1 subfamily selected in Example 1 had superior prediction performance for a treatment response to cancer immunotherapy compared to other random targets, 10 targets in LINE-1 were randomly extracted and the experiment described in Example 1 was repeated.

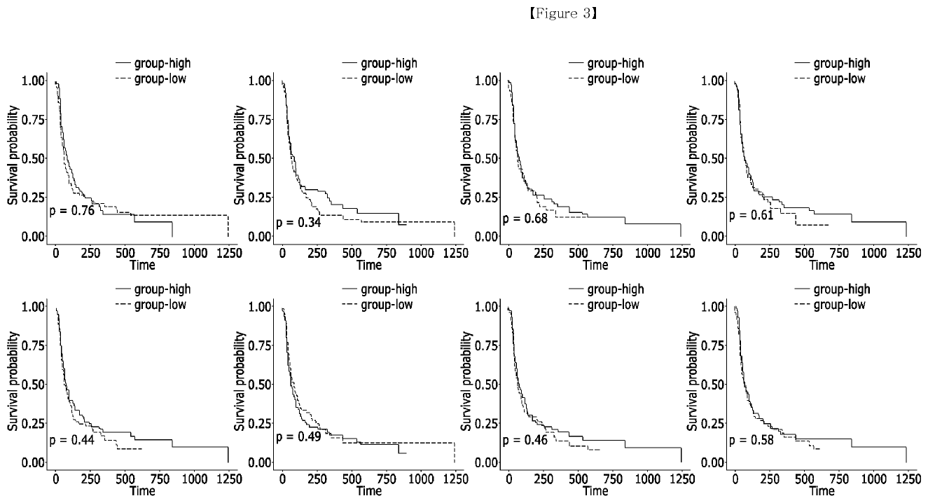

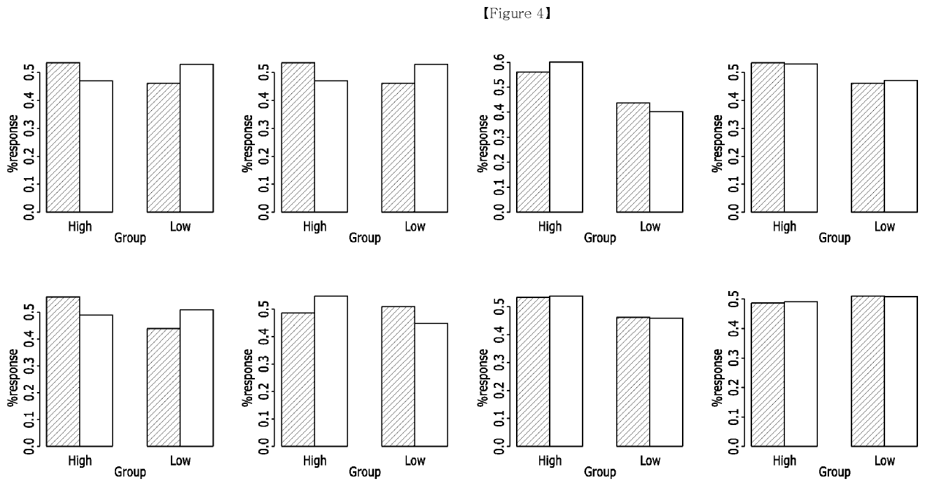

As a result, as can be seen in FIGS. 3 and 4, when the target was randomly selected, no significant results were found in survival analysis and prediction performance analysis of a treatment response to cancer immunotherapy. That is, the LINE-1 subfamily selected in Example 1 had better prediction performance for the treatment response than other targets in LINE-1.

Next, in order to clearly confirm that the LINE-1 subfamily selected in Example 1 has better prediction performance than other targets in LINE-1, 10 targets in LINE-1 were randomly sampled by repeating 1,000 times, and then the p-value distribution for survival analysis and the p-value distribution for the prediction performance analysis of the treatment response to cancer immunotherapy were obtained.

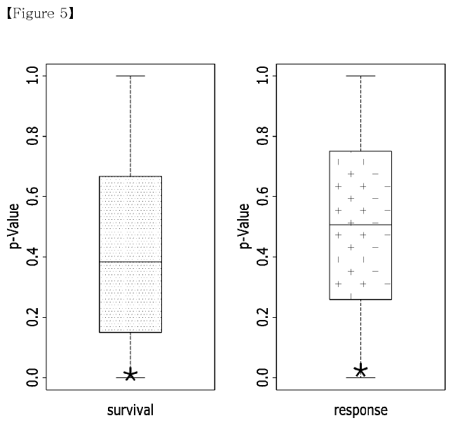

As a result, as illustrated in FIG. 5, it was confirmed that in both the p-value distribution (p-value average 0.418, median 0.383) for survival analysis on the left and the p-value distribution (p-value average 0.502, median 0.506) for the prediction analysis of the treatment response to cancer immunotherapy on the right, when using the methylation values of the LINE-1 subfamily selected in Example 1, the p-value distribution had a significantly low p-value (indicated by a red star, p-value of survival analysis=0.0096, p-value of prediction analysis of the treatment response to cancer immunotherapy=0.026), and the analysis result was significant.

### Example 3. Design of detection PCR primer of LINE-1 subfamily

PCR primer sequences for the 10 LINE-1 subfamilies selected in Example 1 were designed. With the Amplicon size in the range of 100 to 125 bp, forward and reverse primer sets were designed to include the LINE-1 subfamily. Genome Reference Consortium Human Build 37 (GRCh37; hg19) was used as a human genome reference sequence, and Table 2 below showed information on representative positions where 10 LINE-1 subfamilies were mapped to the reference sequence.

TABLE 2

| ID | CHR | TARGET_POSITION | MUTI-HITS |
|---|---|---|---|
| T1 | 19 | 42,007,378 | |
| T2 | 3 | 28,100,733 | |
| T3 | 19 | 55,039,730 | |
| T4 | 11 | 24,239,977 | yes |
| T5 | 11 | 67,350,491 | |
| T6 | 15 | 100,911,974 | |
| T7 | 2 | 195,939,902 | yes |
| T8 | 8 | 12,206,841 | yes |
| T9 | 3 | 94,226,489 | |
| T10 | 11 | 41,668,953 | yes |

Primers were designed to perform PCR amplification for the 10 selected targets. The primers were designed as PCR primers including the targets for a converted sequence, which was fabricated under the assumption that CT was substituted by treating bisulfite to the reference genome sequence. The selected primer sequences were listed in the following table. The primers were designed for the Watson or Crick strand according to the base sequence around the targets.

TABLE 3

| | SEQ | | |
|---|---|---|---|
| Tar- | ID | Prime | |
| get | NO: | ID | Sequence (5′->3′) |
| T1 | 1 | T1_10F | ATTGTAAGTTTTGTTTTTCGAAGTTC |
| | | | GGGGAT |
| | 2 | T1_12F | ATTGTAAGTTTTGTTTTTCGAAGTTTG |
| | | | GGGAT |
| | 3 | T1_14F | ATTGTAAGTTTTGTTTTTTGAAGTTCG |
| | | | GGGAT |
| | 4 | T1_16F | ATTGTAAGTTTTGTTTTTTGAAGTTTG |
| | | | GGGAT |
| | 5 | T1_2F | ATTGTAAGTTTCGTTTTTCGAAGTTC |
| | | | GGGGAT |
| | 6 | T1_4F | ATTGTAAGTTTCGTTTTTCGAAGTTT |
| | | | GGGGAT |
| | 7 | T1_6F | ATTGTAAGTTTCGTTTTTTGAAGTTC |
| | | | GGGGAT |
| | 8 | T1_8F | ATTGTAAGTTTCGTTTTTTGAAGTTTG |
| | | | GGGAT |
| | 9 | T1_15R | TCCCTCTTTTCTTTATTCTCCCGAATC |
| | | | AAA |
| | 10 | T1_16R | TCCCTCTTTTCTTTATTCTCCCAAATC |
| | | | AAA |
| T1 | 11 | T1R_10F | CTACAAACTCCACCTCCCGAAACCCG |
| | | | AAA |
| | 12 | T1R_12F | CTACAAACTCCACCTCCCGAAACCCA |
| | | | AAA |
| | 13 | T1R_14F | CTACAAACTCCACCTCCCAAAACCCG |
| | | | AAA |
| | 14 | T1R_16F | CTACAAACTCCACCTCCCAAAACCCA |
| | | | AAA |
| | 15 | T1R_2F | CTACAAACTCCGCCTCCCGAAACCCG |
| | | | AAA |
| | 16 | T1R_4F | CTACAAACTCCGCCTCCCGAAACCCA |
| | | | AAA |
| | 17 | T1R_6F | CTACAAACTCCGCCTCCCAAAACCCG |
| | | | AAA |
| | 18 | T1R_8F | CTACAAACTCCGCCTCCCAAAACCCA |
| | | | AAA |
| | 19 | T1R_15R | TTTGTTTTTTCGGGTTAAGTTGTTTGT |
| | | | TTAGTT |
| | 20 | T1R_16R | TTTGTTTTTTTGGGTTAAGTTGTTTGT |
| | | | TTAGTT |
| T2 | 21 | T2_1R | CTCACGCTAAAAACTATAAACCGAA |
| | | | ACTATACC |
| | 22 | T2_2R | CTCACGCTAAAAACTATAAACCAAA |
| | | | ACTATACC |
| | 23 | T2_3R | CTCACACTAAAAACTATAAACCGAA |
| | | | ACTATACC |
| | 24 | T2_4R | CTCACACTAAAAACTATAAACCAAA |
| | | | ACTATACC |
| | 25 | T2_4F | GGTGATTATTAAAAAATTAGGAAAT |
| | | | AATAGATGTTGGA |
| T3 | 26 | T3_1R | AACGTACAAATTTACCACATAAATA |
| | | | AACTTATACCC |
| | 27 | T3_2R | AACATACAAATTTACCACATAAATA |
| | | | AACTTATACCC |
| | 28 | T3_2F | TAAATGGGAGAAATTATTTAAAGTTA |
| | | | AGGGGTT |
| T3 | 29 | T3R_1R | GGACGTGTAGGTTTGTTATATAAGTA |
| | | | AATTTGTGTTT |
| | 30 | T3R_2R | GGATGTGTAGGTTTGTTATATAAGTA |
| | | | AATTTGTGTTT |
| | 31 | T3R_2F | TTCCATTCCAAATAAAAAAAATTATC |
| | | | CAAAACC |
| T4 | 32 | T4_1F | AATTTTTAAATGATTTGATGGAGTTG |
| | | | AAAATTATGGG |
| | 33 | T4_1R | ATACTTTCTTCCAATTAATTAAATCC |
| | | | ACTACTAAAAC |
| T5 | 34 | T5R_1R | TAGGTGATTTGTACGTTTCGGTTTTTT |
| | | | AAAGTGTTGGG |
| | 35 | T5R_2R | TAGGTGATTTGTACGTTTTGGTTTTTT |
| | | | AAAGTGTTGGG |
| | 36 | T5R_3R | TAGGTGATTTGTATGTTTCGGTTTTTT |
| | | | AAAGTGTTGGG |
| | 37 | T5R_4R | TAGGTGATTTGTATGTTTTGGTTTTTT |
| | | | AAAGTGTTGGG |
| | 38 | T5R_4F | ATATAACAAACCTACACATCCTCTAC |
| | | | ATATACCC |
| T6 | 39 | T6_1F | AGTAGGGGAAGTTAGATTGAAGGAA |
| | 40 | T6_1R | AAATCAACCTAAATACCCATCAACA |
| | | | ATAAATTAAATA |
| T7 | 41 | T7_1R | CCCGTCACTTTCTAATACACCAATCA |
| | | | AATATAC |
| | 42 | T7_2R | CCCATCACTTTCTAATACACCAATCA |
| | | | AATATAC |
| | 43 | T7_2F | GAAGATAAGAGAAAAAAGTGAAAA |
| | | | GAAATGAATAAAG |
| T8 | 44 | T8_1F | ATTGTAATATTGATGGGTATTTAGGT |
| | | | TGATTTTATG |
| | 45 | T8_1R | TAAAAAATACAATAAACTTATCTACC |
| | | | TTCCAAAACAA |
| T9 | 46 | T9_1F | ATTTAGTTCGCGTTTCGGGGGTTATT |
| | | | TATGTTGT |
| | 47 | T9_2F | ATTTAGTTCGCGTTTTGGGGGTTATT |
| | | | TATGTTGT |
| | 48 | T9_3F | ATTTAGTTCGTGTTTCGGGGGTTATT |
| | | | TATGTTGT |
| | 49 | T9_4F | ATTTAGTTCGTGTTTTGGGGGTTATTT |
| | | | ATGTTGT |
| | 50 | T9_5F | ATTTAGTTTGCGTTTCGGGGGTTATT |
| | | | TATGTTGT |
| | 51 | T9_6F | ATTTAGTTTGCGTTTTGGGGGTTATTT |
| | | | ATGTTGT |
| | 52 | T9_7F | ATTTAGTTTGTGTTTCGGGGGTTATTT |
| | | | ATGTTGT |
| | 53 | T9_8F | ATTTAGTTTGTGTTTTGGGGGTTATTT |
| | | | ATGTTGT |
| | 54 | T9_8R | AATTACTTTTCCTCCACAACCATACT |
| | | | AACAC |
| T10 | 55 | T10_1F | TGGTTTTGTTATTGGATTTGTTTATGT |
| | | | GATGGATTATA |
| | 56 | T10_1R | CAACACATTAAAAAACTTATCCACA |
| | | | ATAATCAAATTAA |

### Example 4. Preparation of NGS library

First, DNA extracted from a patient sample was treated with bisulfite to change unmethylated cytosine into uracil. The bisulfite treatment was performed using an EZ DNA Methylation-Gold kit (Zymo Research) according to the manufacturer's instructions.

Target PCR was performed on the bisulfite-treated DNA using the primers prepared in Example 3 to amplify the 10 LINE-1 subfamilies selected in Example 1. In Example, 10 subfamily targets were divided into two groups (Group 1: T4, T6, T7, T8, and T10, Group 2: T1, T2, T3, T5, and T9) and amplified by a multiplex PCR method. Specifically, PCR primers were synthesized by phosphorylating a 5′ end for NGS adapter ligation and used at a concentration of 200 nM each. A PCR reagent was used with an EpiTect MethyLight PCR kit (Qiagen). Using 1 to 100 ng bisulfite-treated DNA, a target region was amplified by performing total 23 to 29 PCR cycles consisting of once at 95° C. for 5 minutes, at 95° C. for 15 seconds, and at 60° C. for 2 minutes.

The PCR product was purified using 1.8× volume of AMPure XP (Beckman Coulter) beads after mixing PCR reaction solutions of two groups. The purification using magnetic beads was performed according to a manufacturer's using method. An NGS adapter was ligated to the purified PCR product. The NGS adapter was added to the purified PCR product (10 to 100 ng) to be a concentration of 1 μM and ligated using a Quick Ligase kit (NEB). The PCR product was reacted at 25° C. for 30 minutes, and then purified by adding 1× volume of AMPure XP (Beckman Coulter) beads. An adapter-ligated library was amplified using KAPA HiFi HotStart ReadyMix. 200 nM each of the following library amplification primers and KAPA HiFi HotStart ReadyMix were added to the purified sample, and the library was amplified by performing total 7 PCR cycles consisting of once at 98° C. for 45 seconds, at 98° C. for 15 seconds, at 60° C. for 30 seconds, and at 72° C. for 30 seconds, and one PCR cycle at 72° C. for 5 minutes. After amplification, the library was double side purified at 1× to 0.5× (left-right) using AMPure XP (Beckman Coulter) beads.

### <Primers for Library Amplification>

P5:
(SEQ ID NO: 57)
AATGATACGGCGACCACCGAGATCTACAC
P7:
(SEQ ID NO: 58)
CAAGCAGAAGACGGCATACGAGAT

Meanwhile, a library for NGS analysis may be constructed using ready-made AmpliSeq library production reagents (Illumina AmpliSeq Library PLUS, etc.) in addition to the method described above. When using an AmpliSeq Library PLUS (Illumina) library construction reagent, some Ts in the primer sequence of Table 3 were substituted with U for partial digestion by FuPa treatment (Table 4). The library construction method was performed according to a manufacturer's protocol.

TABLE 4

| | SEQ | | |
|---|---|---|---|
| Tar- | ID | Prime | |
| get | NO: | ID | Sequence (5′->3′) |
| T1 | 59 | T1_10F- | ATTGTAAGTTTUGTTTTTCGAAGTUCGG |
| | | U | GGAT |
| | 60 | T1_12F- | ATTGTAAGTTTTGUTTTTCGAAGTTUGG |
| | | U | GGAT |
| | 61 | T1_14F- | ATTGTAAGTTTUGTTTTTTGAAGTUCGG |
| | | U | GGAT |
| | 62 | T1_16F- | ATTGTAAGTTTUGTTTTTTGAAGTTUGG |
| | | U | GGAT |
| | 63 | T1_2F- | ATTGTAAGTTTCGUTTTTCGAAGTUCGG |
| | | U | GGAT |
| | 64 | T1_4F- | ATTGTAAGTTTCGUTTTTCGAAGTTUGG |
| | | U | GGAT |
| | 65 | T1_6F- | ATTGTAAGTTUCGTTTTTTGAAGTUCGG |
| | | U | GGAT |
| | 66 | T1_8F- | ATTGTAAGTTTCGUTTTTTGAAGTTUGG |
| | | U | GGAT |
| | 67 | T1_15R- | TCCCTCTTTTCTUTATTCTCCCGAAUCA |
| | | U | AA |
| | 68 | T1_16R- | TCCCTCTTTTCUTTATTCTCCCAAAUCA |
| | | U | AA |
| T1 | 69 | T1R_10 | CUACAAACTCCACCUCCCGAAACCCGA |
| | | F-U | AA |
| | 70 | T1R_12 | CUACAAACTCCACCUCCCGAAACCCAA |
| | | F-U | AA |
| | 71 | T1R_14 | CUACAAACTCCACCUCCCAAAACCCGA |
| | | F-U | AA |
| | 72 | T1R_16 | CUACAAACTCCACCUCCCAAAACCCAA |
| | | F-U | AA |
| | 73 | T1R_2F | CUACAAACTCCGCCUCCCGAAACCCGA |
| | | -U | AA |
| | 74 | T1R_4F | CUACAAACTCCGCCUCCCGAAACCCAA |
| | | -U | AA |
| | 75 | T1R_6F | CUACAAACTCCGCCUCCCAAAACCCGA |
| | | -U | AA |
| | 76 | T1R_8F | CUACAAACTCCGCCUCCCAAAACCCAA |
| | | -U | AA |
| | 77 | T1R_15 | TTTGTTTTTTCGGGUTAAGTTGTTTGTTT |
| | | R-U | AGUT |
| | 78 | T1R_16 | TTTGTTTTTTTGGGTUAAGTTGTTTGTTT |
| | | R-U | AGUT |
| T2 | 79 | T2_1R- | CTCACGCTAAAAACUATAAACCGAAAC |
| | | U | TAUACC |
| | 80 | T2_2R- | CTCACGCTAAAAACUATAAACCAAAAC |
| | | U | TAUACC |
| | 81 | T2_3R- | CTCACACTAAAAACUATAAACCGAAAC |
| | | U | TAUACC |
| | 82 | T2_4R- | CTCACACTAAAAACUATAAACCAAAAC |
| | | U | TAUACC |
| | 83 | T2_4F- | GGTGATTATTAAAAAATUAGGAAATAA |
| | | U | TAGATGTUGGA |
| T3 | 84 | T3_1R- | AACGTACAAATTUACCACATAAATAAA |
| | | U | CTTAUACCC |
| | 85 | T3_2R- | AACATACAAATTUACCACATAAATAAA |
| | | U | CTTAUACCC |
| | 86 | T3_2F- | TAAATGGGAGAAATTAUTTAAAGTTAA |
| | | U | GGGGUT |
| T3 | 87 | T3R_1R | GGACGTGTAGGTTTGUTATATAAGTAA |
| | | -U | ATTTGTGTUT |
| | 88 | T3R_2R | GGATGTGTAGGTTTGUTATATAAGTAA |
| | | -U | ATTTGTGTUT |
| | 89 | T3R_2F | TTCCATTCCAAAUAAAAAAAATTAUCC |
| | | -U | AAAACC |
| T4 | 90 | T4_1F- | AATTTTTAAATGATTTGAUGGAGTTGAA |
| | | U | AATTAUGGG |
| | 91 | T4_1R- | ATACTTTCTTCCAATUAATTAAATCCAC |
| | | U | TACUAAAAC |
| T5 | 92 | T5R_1R | TAGGTGATTTGTACGTUTCGGTTTTTTA |
| | | -U | AAGTGTUGGG |
| | 93 | T5R_2R | TAGGTGATTTGTACGUTTTGGTTTTTTA |
| | | -U | AAGTGTUGGG |
| | 94 | T5R_3R | TAGGTGATTTGTATGTUTCGGTTTTTTA |
| | | -U | AAGTGTUGGG |
| | 95 | T5R_4R | TAGGTGATTTGTATGTUTTGGTTTTTTA |
| | | -U | AAGTGTUGGG |
| | 96 | T5R_4F | ATATAACAAACCUACACATCCTCTACAT |
| | | -U | AUACCC |
| T6 | 97 | T6_1F- | AGTAGGGGAAGUTAGATUGAAGGAA |
| | | U | |
| | 98 | T6_1R- | AAATCAACCTAAATACCCAUCAACAAT |
| | | U | AAATTAAAUA |
| T7 | 99 | T7_1R- | CCCGTCACTTTCUAATACACCAATCAAA |
| | | U | TAUAC |
| | 100 | T7_2R- | CCCATCACTTTCUAATACACCAATCAAA |
| | | U | TAUAC |
| | 101 | T7_2F- | GAAGATAAGAGAAAAAAGUGAAAAGA |
| | | U | AATGAAUAAAG |
| T8 | 102 | T8_1F- | ATTGTAATATTGATGGGUATTTAGGTTG |
| | | U | ATTTTAUG |
| | 103 | T8_1R- | TAAAAAATACAAUAAACTTATCTACCT |
| | | U | UCCAAAACAA |
| T9 | 104 | T9_1F- | ATTTAGTTCGCGTTUCGGGGGTTATTTA |
| | | U | TGTUGT |
| | 105 | T9_2F- | ATTTAGTTCGCGTTTUGGGGGTTATTTA |
| | | U | TGTUGT |
| | 106 | T9_3F- | ATTTAGTTCGTGTTUCGGGGGTTATTTA |
| | | U | TGTUGT |
| | 107 | T9_4F- | ATTTAGTTCGTGTTTUGGGGGTTATTTA |
| | | U | TGTUGT |
| | 108 | T9_5F- | ATTTAGTTTGCGTTUCGGGGGTTATTTA |
| | | U | TGTUGT |
| | 109 | T9_6F- | ATTTAGTTTGCGTTTUGGGGGTTATTTA |
| | | U | TGTUGT |
| | 110 | T9_7F- | ATTTAGTTTGTGTTUCGGGGGTTATTTA |
| | | U | TGTUGT |
| | 111 | T9_8F- | ATTTAGTTTGTGTTTUGGGGGTTATTTA |
| | | U | TGTUGT |
| | 112 | T9_8R- | AATTACTTTTCCUCCACAACCATACUAA |
| | | U | CAC |
| T10 | 113 | T10_1F- | TGGTTTTGTTATTGGATUTGTTTATGTG |
| | | U | ATGGATTAUA |
| | 114 | T10_1R- | CAACACATTAAAAAACTUATCCACAAT |
| | | U | AATCAAATUAA |

A method for constructing a library using a commercially available kit (Illumina AmpliSeq Library PLUS) was as follows. First, multiplex PCR was performed using the primers in Table 4 above. At this time, the primer concentration was set to 200 nM each, and 10 targets were divided into 1 or 2 groups and amplified by a multiplex PCR method. The PCR primer set and AmpliSeq HiFi Mix were added to 1 to 100 ng bisulfite-treated DNA, and the target region was amplified by performing total 26 to 32 PCR cycles consisting of once at 99° C. for 2 minutes, at 99° C. for 15 seconds, and at 60° C. for 2 minutes.

1/10 vol of a FuPa Reagent was added to the PCR product and reacted at 50° C. for 10 minutes, at 55° C. for 10 minutes, and at 60° C. for 20 minutes. Thereafter, the PCR product was added with a Switch solution and mixed well, added with an adapter and DNA ligase, and reacted at 22° C. for 30 minutes, 68° C. for 5 minutes, and 72° C. for 5 minutes, and then the adapter was attached to the PCR product.

The ligation product was purified by adding 1× AMPure XP (Beckman Coulter) beads. In the last step of purification, 45 μL of 1× Lib Amp Mix and 5 μL of 10× Library Amp Primers were added to the washed and dried beads instead of a solution for elution. The library was amplified by performing total 7 cycles consisting of once at 98° C. for 2 minutes, at 98° C. for 15 seconds, and at 64° C. for 1 minute. After amplification, the library was double side purified at 1.2× to 0.5× (left-right) using AMPure XP (Beckman Coulter) beads.

The library constructed through the process was quantified by real-time PCR using a KAPA library quantification kit (Kapa Biosystems) and then subjected to NGS reaction.

### Example 5. NGS Analysis

From a BCL file produced by an NGS machine, an NGS sequence produced for each specimen was separated (demultiplexed) into a fastq file form using a Bcl2fastq program (ver2.20.0.422). There were prepared a bisulfite standard genome sequence reflecting a C-to-T (cytosine base was changed to thymine base) or G-to-A (guanine base was changed to adenine base) change capable of occurring due to bisulfite treatment by applying the bismark program (ver 0.22.3) based on the UCSC hg19 human genome standard sequence downloaded from a public database and an index file for sequence mapping. An illumina universal adapter sequence was trimmed from the fastq file for each specimen and then aligned to the prepared bisulfite standard genome sequence using the bismark program. The sequences aligned to a Watson strand and a Crick strand were separated from the Sequence alignment map (SAM) file, and a bam-count-table containing information on the number of A, T, G, and C bases at each LINE-1 subfamily target position was obtained using the bam-readcount program (ver 0.8.0). From this count-table, the DNA methylation level of the Long Interspersed Nuclear Element-1 (LINE-1) subfamily was calculated for each position of each of the 10 subfamilies.

An estimate of the methylation level of the LINE-1 subfamily may be represented as a beta value, and the beta value may represent the degree of methylation in the range of 0 to 1. For example, 0 means that the corresponding CpG locus is not completely methylated, and 1 means that the corresponding CpG locus is completely methylated.

The beta values at the target positions of the 10 LINE-1 subfamilies selected in Example 1 were integrated and taken as one iMethyl score for each specimen. The iMethyl score calculation and cut-off values were selected using logistic regression analysis based on previous research of applying machine learning from beta values and treatment response data in a lung cancer cohort treated with cancer immunotherapy, and the iMethyl score cut-off value for predicting the treatment response in the lung cancer cohort was 0.37. The optimal combination of probes and variables was found through 3-fold validation using a logistic regression method on 90 patients who had cancer immunotherapy response data and beta value data of T1 to T10, and Equation for obtaining the final iMethyl score from beta values of each probe was as follows.

$$iMethyl\_score = exp ( methyl\_score ) / ( 1 + exp ( methyl\_score ) ) \\ methyl\_score = {\sum }_{i = 1}^{10} {a}_{i} * {Ti}_{beta} + b$$

Ti_{beta} was a beta value at each probe position, and aᵢ and b were variable values obtained through machine learning.

### Example 6. Comparison of Methylation Values for Infinium Methylation 450k Microarray and NGS

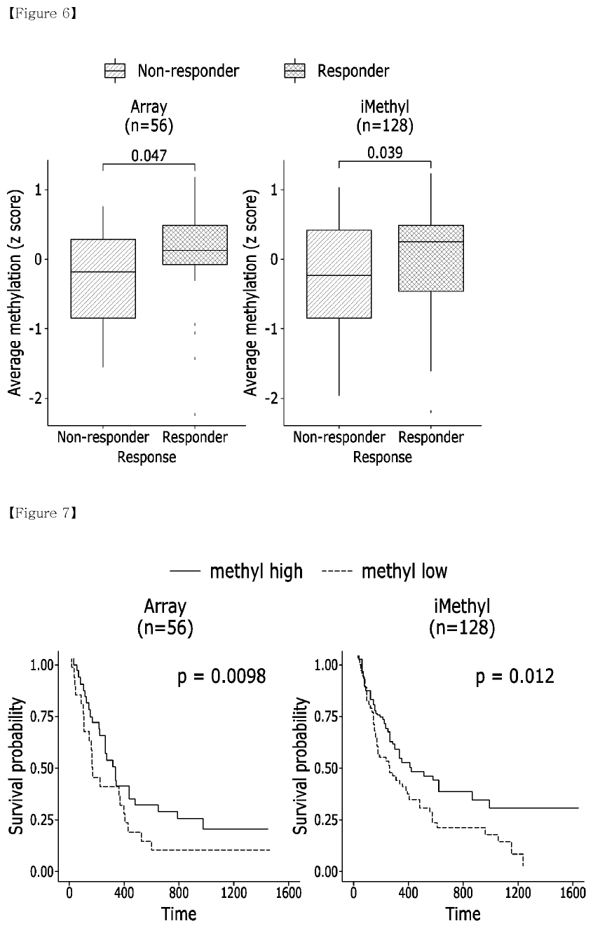

Next, in order to evaluate the prediction performance of the treatment response of the LINE-1 subfamily selected in Example 1 for lung cancer patients with a history of immune checkpoint inhibitor therapy, a difference in distribution of methylation values between the responder and non-responder groups was confirmed. As a result, as shown in FIG. 6, it was confirmed that the methylation value obtained by Infinium Methylation 450k microarray was p-value=0.047, while the iMethyl score obtained by the method described in Example 5 was p-value=0.039, which exhibited better prediction performance.

Next, a difference in survival probability between a response prediction group (a patient group predicted to have a high treatment response) and a non-response prediction group (a patient group predicted to have a low treatment response) to an immune checkpoint inhibitor was analyzed using a Kaplan-Meier estimation technique based on the methylation value obtained by Infinium Methylation 450k microarray or the iMethyl score obtained by the method described in Example 5 above.

As a result, as shown in FIG. 7, it was confirmed that the iMethyl score (p-value=0.012) for 10 LINE-1 subfamilies by the method described in Example 5 above showed superior prediction performance than the value (p-value=0.098) for the Infinium Methylation 450k microarray method.

Factors widely known for predicting a response to an immune checkpoint inhibitor includes tumor mutation burden (TMB), neoantigen load (NeoAg), and the presence or absence of PD-L1 (SP263) expression. Based on each predictive factor, the difference in survival probability was analyzed by dividing the groups into a response prediction group and a non-response prediction group to an immune checkpoint inhibitor using the Kaplan-Meier estimation technique in the same manner, and through a Log-Rank test, a statistical significance of a difference in survival curves between the response prediction group and the non-response prediction group was derived as P-value. In order to make a more meaningful comparison between response prediction factors to an immune checkpoint inhibitor, patient samples were randomly sampled by 1,000 repetitions for each factor, and the p-value distributions of survival analysis were compared.

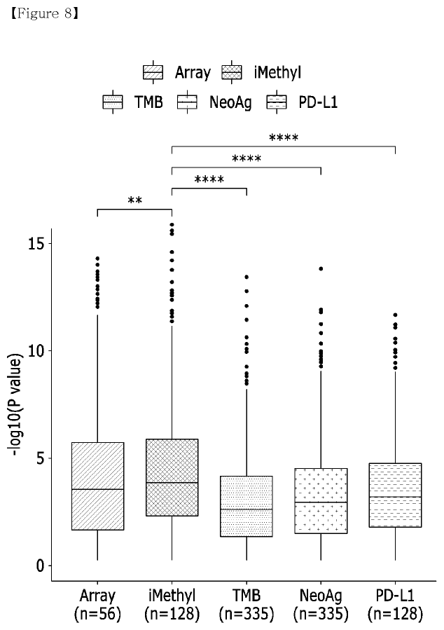

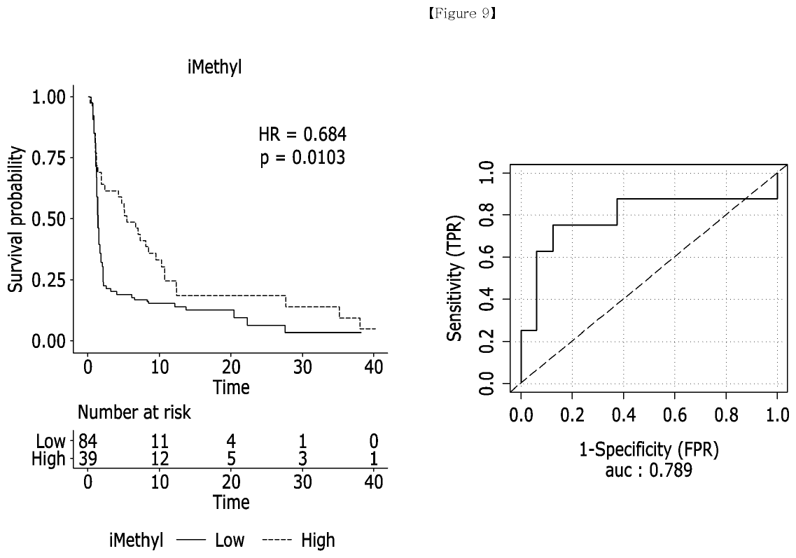

As a result, as can be seen in FIG. 8, it was confirmed that the iMethyl score for the LINE-1 subfamily was predicted better than other existing factors to the immune checkpoint inhibitor, and especially, the iMethyl score calculated using the method described in Example 5 showed the highest prediction performance.

### Example 7. Verification of prediction performance for treatment response in lung cancer patients

Methylation levels, that is, beta values for 10 LINE-1 subfamilies were analyzed by the method (iMethyl) described in Example 5 above using DNA extracted from tissues or FFPE specimens of patients with respect to a total of 123 lung cancer patients with a history of anti-PD1 or anti-PD-L1 immune checkpoint inhibitor therapy.

The Kaplan-Meier estimation technique was used to analyze a difference in survival probability between a response prediction group (a patient group predicted to have a high treatment response) and a non-response prediction group (a patient group predicted to have a low treatment response) to an immune checkpoint inhibitor based on the iMethyl score. The survival probability was basically based on progression free survival (PFS). The statistical significance of the difference in survival curve between the response prediction group and the non-response prediction group was derived as P-value through the Log-Rank test, and a hazard ratio (HR) was calculated using a Cox's proportional hazard model. The final prediction performance was evaluated through an Area Under a Curve (AUC) value of a Receiver Operating Characteristic (ROC) curve using sensitivity and false positive rate. The statistical significance of a difference between groups in categorical variables was analyzed using a Fisher's exact test (comparison of 2 Groups). The statistical significance of a difference in average or median values of continuous variables was analyzed using a Mann-Whitney U test.

As a result of comparing survival probabilities of two groups divided into a high predicted iMethyl score group (response prediction group, high, 39 patients) and a low predicted iMethyl score group (non-response prediction group, low, 84 patients), as shown in FIG. 9, at a statistically significant level of p-value=0.0103, the survival probability of the non-response prediction group was HR=0.684 times lower than that of the response prediction group, and the AUC was confirmed as 0.789, which showed outstanding prediction performance.

### Example 8. Sample-Specific Correlation for Methylation Values of LINE-1 Subfamilies

First, the distribution of methylation values for blood cells (PBMC), cell free DNAs (cfDNA), and an FFPE specimen (Tissue) of tumor tissue for 50 breast cancer patients was confirmed using the 10 LINE-1 subfamilies selected in Example 1 above. The methylation values were obtained using the NGS method described in Example above.

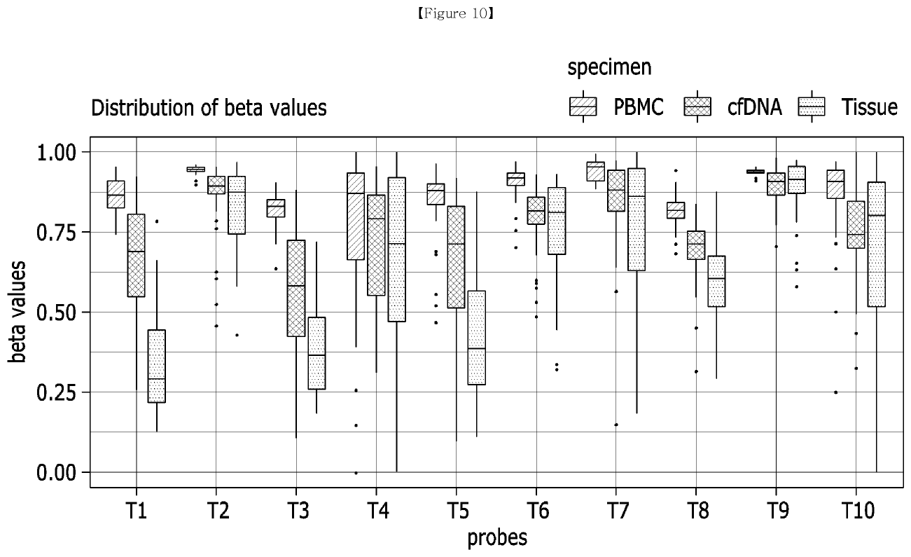

As a result, as shown in FIG. 10, in each subfamily, overall, the cell-free DNA (cfDNA) had a methylation value distribution that tended to be more similar to tumor tissue than blood cells (PBMC), and the cfDNA and tumor tissue had a lower methylation value distribution than the blood.

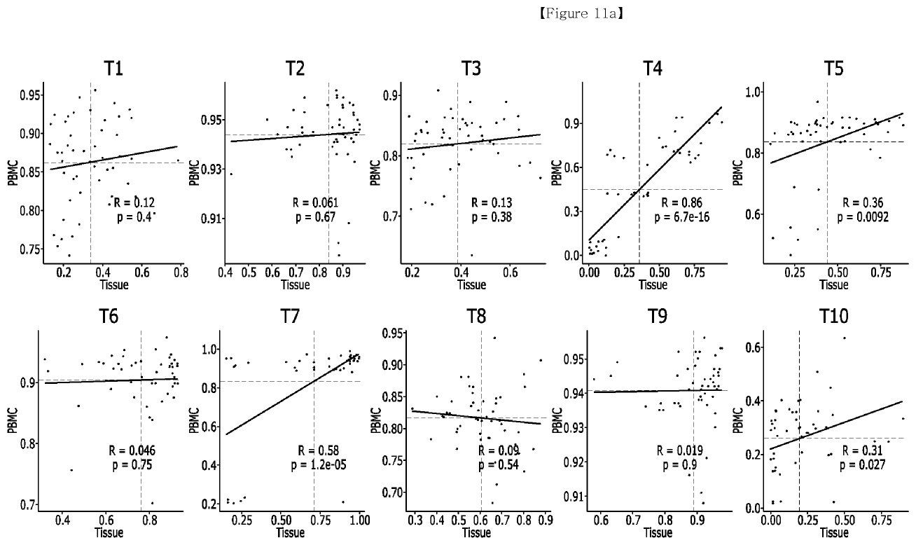

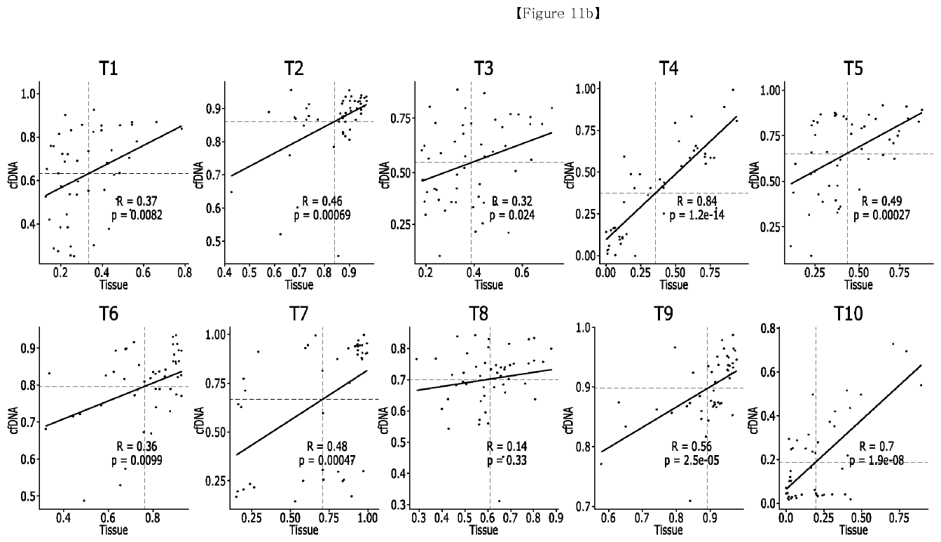

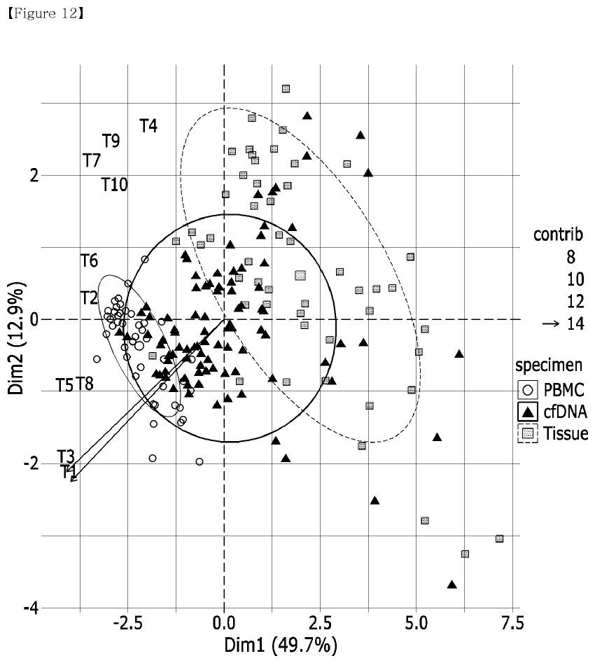

Next, correlations were confirmed between the blood and the FFPE specimen of tumor tissue, and between cfDNA and the FFPE specimen of tumor tissue, for each of the 10 LINE-1 subfamilies. As can be seen in FIGS. 11A and 11B, it was confirmed that overall, the correlation between blood and tumor tissue was not significant, while the cfDNA and tumor tissue showed a strong positive correlation in most cases.

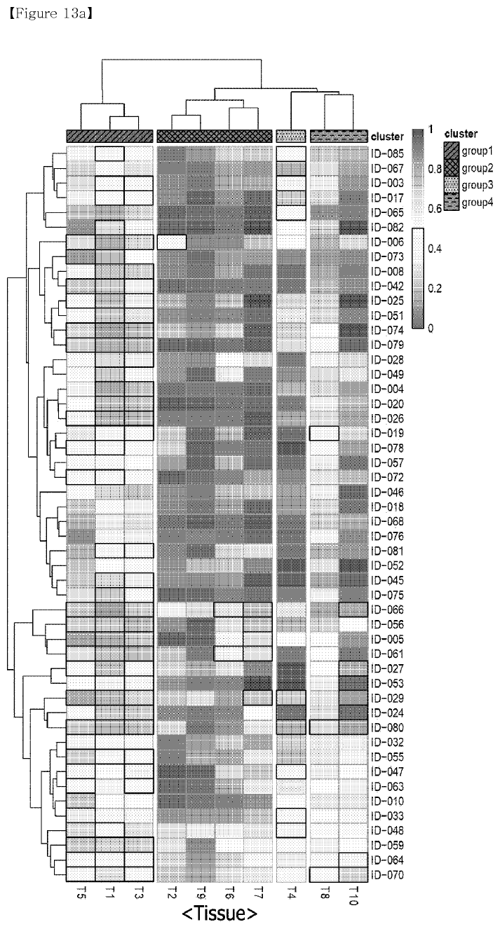

In addition, in the entire LINE-1 subfamilies, dimensionality was reduced using Principal Component Analysis (PCA) to more clearly visualize the correlation between cfDNA and tumor tissue, as shown in FIG. 12. As shown in FIG. 12, it was confirmed that the blood was distributed separately from tumor tissue, while cfDNA was distributed very similarly to tumor tissue. Therefore, it was confirmed that the correlation between cfDNA and tumor tissue was clearer.

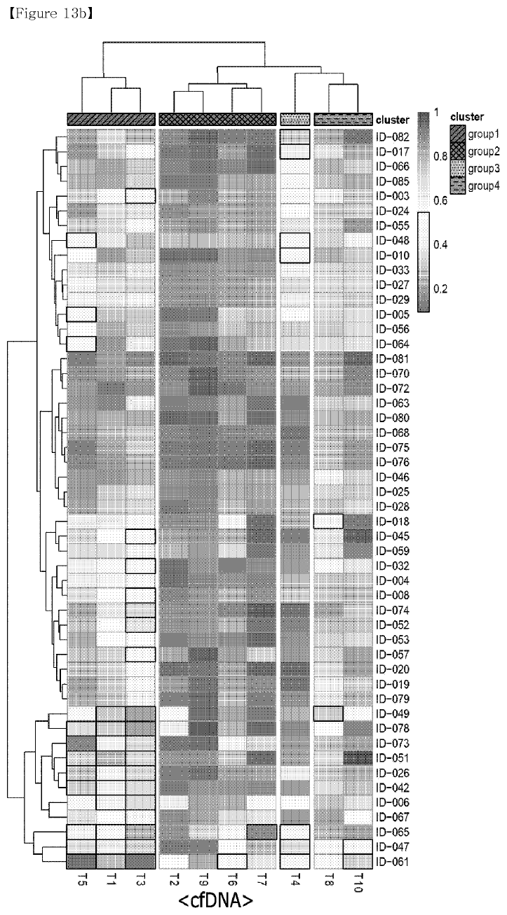

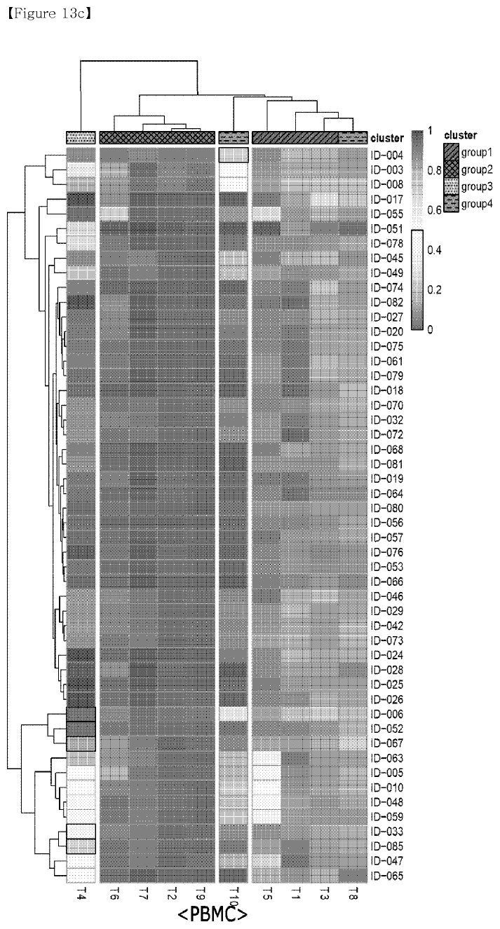

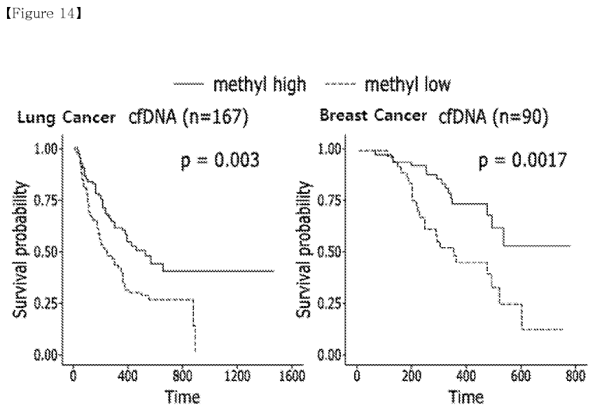

In addition, in order to confirm the clustering tendency and association between LINE-1 subfamilies, hierarchical clustering was performed on LINE-1 subfamilies in blood cells (PBMC), cfDNA, and tumor tissue (Tissue), respectively. As a result, as shown in FIGS. 13A to 13C, tumor tissue (Tissue) and cfDNA were identically clustered to [{T5, T1, T3}/{T2, T9, T6, T7}/{T4}/{T8, T10}]. Each cluster was represented as group 1 to group 4. On the other hand, the blood (PBMC) was clustered as [{T4}/{T6, T7, T2, T9}/{T10}/{T5, T1, T3, T8}] to show a different pattern from tumor tissue.

### Example 9. Verification of Prediction Performance for Treatment Response Using cfDNA in Lung Cancer and Breast Cancer Patients

With respect to a total of 167 lung cancer patients and 90 breast cancer patients with a history of immune checkpoint inhibitor therapy, the methylation levels, that is, beta values of 10 LINE-1 subfamilies were analyzed by the method (iMethyl) described in Example 5 above using the cell-free DNA (cfDNA) of the patients.

The Kaplan-Meier estimation technique was used in the same manner as the method described in Example 7 above to analyze a difference in survival probability between a response prediction group (a patient group predicted to have a high treatment response) and a non-response prediction group (a patient group predicted to have a low treatment response) to an immune checkpoint inhibitor based on the iMethyl score.

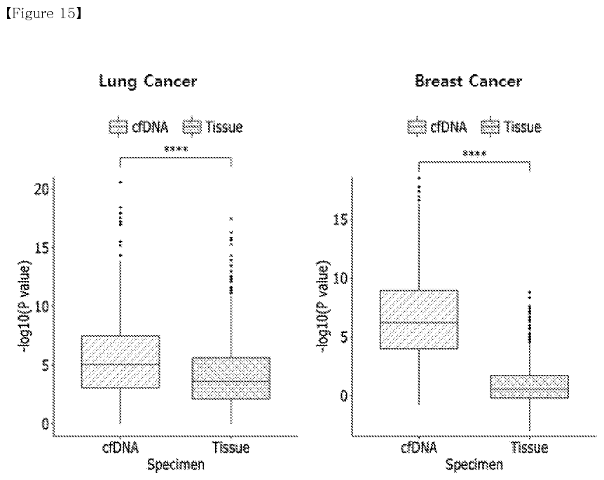

As a result of comparing the survival probabilities of two groups divided into a high predicted iMethyl score group (response prediction group, high) and a low predicted iMethyl score group (non-response prediction group, low), as shown in FIG. 14, at a statistically significant level of p-value=0.003 for lung cancer and p-value=0.0017 for breast cancer, the survival probability of the non-response prediction group was predicted to be lower than that of the response prediction group.

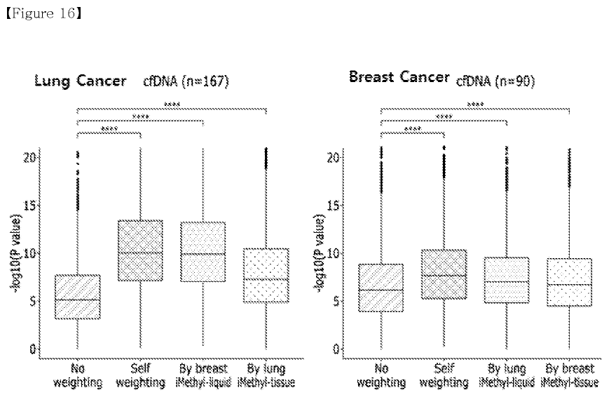

Next, the response prediction performance of the immune checkpoint inhibitor was compared according to the methylation values obtained from cfDNA and the methylation values obtained from tumor tissue. For statistical significance, patient samples were randomly sampled, each repeated 1,000 times, and survival analysis p-values were obtained. As can be seen in FIG. 15, survival analysis using cfDNA showed better prediction performance than survival analysis using tumor tissue in both lung cancer patients and breast cancer patients.

Additionally, in order to make the prediction of a response to an immune checkpoint inhibitor more meaningful, the iMethyl scores were calculated by giving different weights to each of the 10 LINE-1 subfamilies selected in Example 1 (Weighting). The weights that showed the best prediction performance in each of a group of 167 cfDNA lung cancer patients and a group of 90 cfDNA breast cancer patients were applied (self-weighting), or the weights that showed the best prediction performance in the other patient groups were cross-applied (cross weighting). As a result, as shown in FIG. 16, the iMethyl scores calculated with weights showed more significant prediction performance than the existing iMethyl scores. In particular, it was confirmed that the prediction performance was also further improved by cross-applying weights from tumor tissue or cross-applying weights from other tumor cfDNA.

Summarizing the results, it can be seen that since the methylation value obtained from cfDNA shows a high correlation with the methylation value of tumor tissue, a treatment response to cancer immunotherapy can be predicted with higher accuracy using cfDNA.

Although the embodiments have been described with limited drawings as described above, those skilled in the art can apply various technical modifications and variations based on the above. For example, even if the described techniques are performed in a different order than the described method, and/or the described components are combined or joined in a different form than the described method or are replaced or substituted by other components or equivalents, adequate results can be achieved.

Therefore, other implementations, other embodiments, and equivalents of the claims also fall within the scope of the claims described below.

1. A method for providing information for predicting a response to cancer therapy comprising:
obtaining information on a DNA methylation level of a long interspersed nuclear element-1 (LINE-1) subfamily from an isolated sample of a cancer patient, wherein the LINE-1 subfamily is at least one selected from the group consisting of LIPA12_5end, LIHS_5end, LIPA10_3end, LIP2_5end, LIPA11_3end, LIP2_5end, LIPA13_3end, LIPA11_3end and LIP1_orf2.

2. The method for providing information for predicting the response to cancer therapy of claim 1, wherein the obtaining of the information on the DNA methylation level of the LINE-1 subfamily from the isolated sample of the cancer patient includes
detecting methylated and unmethylated DNAs of the LINE-1 subfamily; and
calculating an estimate of the methylation level of the LINE-1 subfamily using a ratio between the methylated and unmethylated DNAs.

3. The method for providing information for predicting the response to cancer therapy of claim 2, wherein when there are the several LINE-1 subfamilies, the method includes calculating a score by multiplying the estimate of each methylation level by a weight coefficient value and then adding the values.

4. The method for providing information for predicting the response to cancer therapy of claim 2, wherein the detecting of the methylated and unmethylated DNAs includes performing next generation sequencing.

5. The method for providing information for predicting the response to cancer therapy of claim 1, wherein the cancer therapy is cancer immunotherapy.

6. The method for providing information for predicting the response to cancer therapy of claim 1, wherein the sample is cell free DNA.

7. The method for providing information for predicting the response to cancer therapy of claim 1, wherein the cancer is any one cancer selected from the group consisting of melanoma, bladder cancer, esophageal cancer, glioma, adrenal cancer, sarcoma, thyroid cancer, colorectal cancer, prostate cancer, head and neck cancer, urothelial cancer, stomach cancer, pancreatic cancer, liver cancer, testicular cancer, ovarian cancer, endometrial cancer, cervical cancer, brain cancer, breast cancer, kidney cancer, and lung cancer.
